(19) United States
(12) Patent Application Publication
(10) Pub. No.: US 2023/0210987 A1
(43) Pub. Date: Jul. 6, 2023

(54) ADJUVANT AND VACCINE CONTAINING ADJUVANT

(71) Applicant: The University of Tokyo, Tokyo (JP)

(72) Inventors: Yoshihiro KAWAOKA, Tokyo (JP); Huapeng FENG, Tokyo (JP); Tokiko WATANABE, Tokyo (JP); Makoto YAMASHITA, Tokyo (JP)

(73) Assignee: The University of Tokyo, Tokyo (JP)

(21) Appl. No.: 18/119,973

(22) Filed: Mar. 10, 2023

Related U.S. Application Data
(63) Continuation of application No. 17/041,147, filed on Dec. 15, 2020.

(30) Foreign Application Priority Data
Mar. 27, 2018 (JP) 2018-059532

(51) Int. Cl. A61K 39/145; A61K 39/39; C12N 7/00; A61K 39/295; A61K 39/12

(52) U.S. Cl. CPC A61K 39/145; A61K 39/39; C12N 7/00; A61K 39/295; A61K 39/12; C12N 2760/16034; C12N 2760/16071; A61K 2039/70

(57) ABSTRACT

The present invention is intended to provide an adjuvant having high safety to living bodies and an action to sufficiently reinforce immune function, and a vaccine comprising the adjuvant. Specifically, the present invention relates to 34 novel adjuvant candidate compounds, which have been identified by screening 145 food additives and 51 injection additives, using, as indicators, an increase in the antibody titer against influenza virus and a protective effect against infection with influenza virus, and then selecting those having the function of increasing the antiviral antibody titer in blood and the protective effect against viral infection. In addition, the present invention also relates to a vaccine comprising these adjuvant candidate compounds.

### TECHNICAL FIELD

The present invention relates to an adjuvant and a vaccine comprising the same.

### BACKGROUND ART

“Adjuvant” is a term originally derived from the term of Latin “adjuvare (to help),” and the adjuvant is also referred to as an antigenic reinforcement, an immunostimulator, or the like. The adjuvant acts to stimulate dendritic cells in an immune system, and in particular, in a humoral immune mechanism, and to enhance generation of antibodies or immune function by T cells. Thus, the adjuvant is used for the purpose of enhancing the immunogenicity of a vaccine by being administered together with the vaccine.

In general, the effects of a vaccine are evaluated based on the immunogenicity, safety and production costs thereof. The effects of an adjuvant also tend to be evaluated in the same manner as that for the vaccine. To date, precipitation adjuvants such as sodium hydroxide, aluminum hydroxide, calcium phosphate and aluminum phosphate, oil-based adjuvants such as liquid paraffin, lanolin and freund, etc. have been known. Among others, since an aluminum hydroxide gel adjuvant (alum) was discovered in 1920s, alum has been most widely used as an adjuvant for human vaccines. In recent years, MF59 (registered trademark) and AS03, which comprise an oil-in-water type emulsion of squalene as a main component, have been approved as adjuvants for human influenza vaccines (Non Patent Literature 1).

By the way, the safety of the conventionally used adjuvants has not been necessarily secured. For example, it has been reported that the incidence rate of narcolepsy is increased in children inoculated with an influenza vaccine comprising a squalene adjuvant, and thus, the concerns regarding the safety of the adjuvant vaccine have been raised (Non Patent Literature 2 and Non Patent Literature 3). Moreover, it has also been reported that a squalene adjuvant causes local and systemic reactogenicity (Non Patent Literature 4 and Non Patent Literature 5).

As mentioned above, needless to say, an adjuvant is an extremely important agent that exhibits the function of reinforcing the immunogenicity of a vaccine and prevents infectious disease, autoimmune disease and the like. However, it is also a reality that problems regarding the safety thereof are pointed out. Thus, the development of an adjuvant having high safety and excellent immunogenicity-enhancing effect is still one of important objects to be achieved in the field of immunotherapy.

### CITATION LIST

### Non Patent Literature

Non Patent Literature 1: Weir et al., Influenza and other respiratory viruses 10: 354-360, 2016
Non Patent Literature 2: Szakacs et al., Neurology 80: 1315-1321, 2013
Non Patent Literature 3: Nohynek et al., PloS one 7: e33536, 2012
Non Patent Literature 4: Petrovsky et al., Drug safety 38: 1059-1074, 2015
Non Patent Literature 5: Fox et al., Expert review of vaccines 12: 747-758, 2013

### SUMMARY OF INVENTION

### Technical Problem

In consideration of the aforementioned circumstances, it is an object of the present invention to develop an adjuvant that is a compound whose safety to living bodies has been confirmed and which has an action to sufficiently reinforce immune function.

### Solution to Problem

The present inventors have screened 145 food additives and 51 injection additives, using, as indicators, an increase in the antibody titer against influenza virus and the effect of protecting against infection with influenza virus. As a result, the present inventors have found that 41 compounds derived from the food additives and 21 compounds derived from the injection additives have the function of increasing the antiviral antibody titer in blood and a protective effect against viral infection. The present invention has been completed based on the aforementioned findings.

Specifically, the present invention includes the following (1) to (4).

(1) An adjuvant comprising one or more selected from the group consisting of norbixin, neotame, (R)-(+)-citronellal, crocin, γ-undecalactone, abietic acid, brilliant blue FCF, carminic acid, (+/−)-citronellol, fast green FCF, geranyl formate, hydroxycitronellal, indigo carmine, iron (II) gluconate n-hydrate, isoeugenol, methyl anthranilate, naringin, terpineol, hydroxypropyl cellulose, ethanol, sodium benzoate, sodium sulfite, EMANON CH-25, sodium thiosulfate, sodium hydrogen sulfite, ammonium acetate, EMANON CH-60K, EMANON CH-40, sodium D-gluconate, potassium chloride, sodium acetate, sodium bromide, 1,1,1-trichloro-2-methyl-2-propanol hemihydrate, and xylitol.
(2) A mucosal vaccine adjuvant comprising one or more selected from the group consisting of calcium glycerophosphate hydrate, rutin hydrate, sepiolite, β-D-glucan, riboflavin, saponin, and Poly(I:C).
(3) A vaccine comprising the adjuvant according to the above (1) or (2) and an antigen.
(4) The vaccine according to the above (3), wherein the antigen is at least one selected from the group consisting of: viruses, bacteria, parasites, fungi, rickettsiae, chlamydia, prion, cancer cells, and molecules derived from these; cancer antigens; autoimmune disease-related antigens; and allergy-related antigens.

### Advantageous Effects of Invention

The adjuvant according to the present invention increases generation of antibodies against antigens and exhibits an excellent protective function against infection with virus. Moreover, since the safety of the adjuvant according to the present invention to living bodies has already been confirmed, it is extremely unlikely that the adjuvant according to the present invention would cause side effects.

A vaccine comprising the adjuvant according to the present invention is safe to living bodies, for example, in prevention of infectious disease, and has a high infection protective effect.

### BRIEF DESCRIPTION OF DRAWINGS

FIG. 1 shows the influence (1) of adjuvant candidate compounds on the antibody titer against the Ebola GP protein. A: EMANON CH-60K, B: hydroxypropyl cellulose, and C: polyoxyethylene polyoxypropylene glycol)(160E.O.) (30P.O.).

FIG. 2 shows the influence (2) of adjuvant candidate compounds on the antibody titer against the Ebola GP protein. D: calcium glycerophosphate hydrate, E: sodium chondroitin sulfate, and F: riboflavin.

FIG. 3 shows confirmation of the effects of nasal (mucosal) vaccine adjuvants. Calcium glycerophosphate (A and B) and rutin hydrate (C and D) were formulated into nasal vaccine adjuvants, and the nasal vaccine adjuvants were then administered to mice. The results obtained by examining the influence of the adjuvants on the body weight (A and C) and the survival rate (B and D) of the mice are shown in FIG. 3.

### DESCRIPTION OF EMBODIMENTS

A first embodiment of the present invention relates to an adjuvant comprising one or more selected from the group consisting of abietic acid, brilliant blue FCF, carminic acid, (+/−)-citronellol, (R)-(+)-citronellal, crocin, fast green FCF, geranyl formate, hydroxycitronellal, indigo carmine, iron (II) gluconate n-hydrate, isoeugenol, methyl anthranilate, naringin, neotame, norbixin, terpineol, γ-undecalactone, calcium glycerophosphate hydrate, calcium sorbate, β-carotene, sodium chondroitin sulfate, β-D-glucan, monoammonium glycyrrhizinate, hesperidin, isoquinoline, pectin, polysorbate 20, polysorbate 60, polysorbate 80, rutin, rutin hydrate, theobromine, β-cyclodextrin, sodium poly-L-γ-glutamate, polyvinylpyrrolidone (molecular weight: 3,600,000), pullulan, riboflavin, saponin, sepiolite, sodium alginate 80 to 120, dextran 40, gum Arabic, polyethylene glycol 4,000, polyoxyethylene polyoxypropylene glycol) (160E.O.) (30P.O.), RHEODOL AO-15V, ammonium acetate, EMANON CH-25, EMANON CH-40, EMANON CH-60K, ethanol, sodium D-gluconate, hydroxypropyl cellulose, potassium chloride, sodium acetate, sodium benzoate, sodium hydrogen sulfite, sodium bromide, sodium sulfite, sodium thiosulfate, zinc oxide, 1,1,1-trichloro-2-methyl-2-propanol hemihydrate, and xylitol (hereinafter also referred to as “the adjuvant according to the present invention”). CAS Numbers of individual compounds and examples of source companies are summarized in Table 1 and Table 5. The above-described compounds may each be in the form of the salts thereof, or their solvates or hydrates.

The “adjuvant” according to the present invention has the same definitions as those referred to as an “antigenic reinforcement,” an “immunostimulator,” or the like. In the present technical field, the adjuvant according to the present invention is used for the ordinary intended use of the aforementioned agents. Moreover, the administration method of a vaccine comprising the adjuvant according to the present invention is not particularly limited, and any method may be applied. Examples of the administration method may include administration involving an intramuscular injection and nasal (mucosal) administration.

The adjuvant according to the present invention comprises, for example, approximately 0.01% to 99.99% by weight of one or more of the aforementioned compounds, although the amount of the compound(s) is not limited thereto. Moreover, the present adjuvant may be any of the aforementioned compounds themselves.

The adjuvant according to the present invention may comprise a component other than the aforementioned compounds, which does not inhibit the functions of the aforementioned compounds as adjuvants. Examples of such a component may include a stabilizer, a pH adjuster, a preservative, an antiseptic, and a buffer.

Moreover, the adjuvant according to the present invention may comprise a component other than the aforementioned compounds, which has been known to be comprised in existing adjuvants and to have immunostimulatory activity. Examples of such a component may include, but are not limited to, aluminum hydroxide, squalene, mineral oil, paraffin oil, nucleic acid, and a trehalose derivative.

The adjuvant according to the present invention can be used to all organisms having an immune mechanism. Examples of such organisms may include, but are not limited to: vertebrates (mammals (a human, a mouse, a rat, a rabbit, a llama, a camel, sheep, a goat, etc.), birds (a chicken, etc.), reptiles, amphibians, and fishes); and invertebrates (arthropods (insects, crustaceans, arachnids, and myriapoda), mollusks, etc.).

A second embodiment of the present invention relates to a vaccine comprising the adjuvant according to the present invention and an antigen (hereinafter also referred to as “the vaccine according to the present invention”).

The antigen is not particularly limited, as long as it causes an immune response. Examples of the antigen may include viruses, bacteria, parasites, fungi, rickettsiae, chlamydia, prions and cancer cells, and molecules derived from these (e.g., proteins, nucleic acids, sugars, and lipids); biomolecules other than those described above, such as proteins, nucleic acids, sugars and lipids; and disease-related antigens (cancer antigens, autoimmune disease-related antigens, and allergy-related antigens, etc.).

Viruses, bacteria, parasites, fungi, rickettsiae, chlamydia and the like, which are attenuated, may also be used as antigens (live vaccines). Otherwise, viruses, bacteria, parasites, fungi, rickettsiae, chlamydia and the like, which are inactivated, may also be used as antigens (inactivated vaccines). In addition, toxoid, capsid, a surface-presenting protein and the like, which are derived from these, may also be used as antigens.

Moreover, as antigens derived from cancer cells, what is called, cancer antigens, such as proteins specifically expressed in cancer cells (e.g., PSA (prostate-specific antigen) of prostate cancer, etc.), may be used.

In this context, the virus is not particularly limited, as long as it is infected into animals including humans and causes disease to them. Examples of the virus may include influenza virus, Ebola virus, nipah virus, adenovirus, papillomavirus, human immunodeficiency virus, hepatitis virus (type A, type B, type C, type D, type E, type F, type G, etc.), measles virus, rubella virus, poliovirus, rotavirus, norovirus, sapovirus, enterovirus, rabies virus, yellow fever virus, varicella-zoster virus, mumps virus, cytomegalovirus, coronavirus, polyomavirus, herpesvirus, Japanese encephalitis virus, dengue virus, Marburg virus, parvovirus, Lassa virus, Hantavirus, Thogoto virus, Dhori virus, Newcastle virus, Togavirus, paramyxovirus, orthomyxovirus, poxvirus, reovirus, and foot-and-mouth disease virus.

The above-described bacteria are not particularly limited, as long as they are infected into animals including humans and cause disease to the animals. Examples of the bacteria may include Streptococcus, Staphylococcus aureus, Enterococcus, Listeria monocytogenes, pathogenic Escherichia coli, Bordetella pertussis, Corynebacterium diphtheriae, Klebsiella pneumoniae, Proteus, Meningococcus, Pseudomonas aeruginosa, Serratia marcescens, Gonococcus, Enterobacter, Citrobacter, Mycoplasma, Clostridium, Mycobacterium tuberculosis, Vibrio cholerae, Yersinia pestis, Shigella, C. tetani, Anthrax, Treponema pallidum, Legionella, Leptospira, Helicobacter pylori, Borrelia, and Haemophilus influenzae.

The above-described parasites are not particularly limited, as long as they are infected into animals including humans and cause disease to the animals. Examples of the parasites may include Malaria parasites, Toxoplasma gondii, Leishmania, Trypanosoma, Cryptosporidium, Echinococcus, Schistosomiasis, Filaria, and roundworms.

The above-described fungi are not particularly limited, as long as they are infected into animals including humans and cause disease to the animals. Examples of the fungi may include Candida fungus, Aspergillus fungus, Cryptococcus fungus, Histoplasma fungus, ringworm fungus, Pneumocystis fungus, and Coccidioidomycosis fungus.

The above-described cancer is not particularly limited, as long as it is developed in animals including humans. Examples of the cancer may include leukemia, malignant lymphoma, nerve tumor, melanoma, bone tumor, brain tumor, head and neck cancer, tongue cancer, thyroid cancer, pharyngeal cancer, laryngeal cancer, esophageal cancer, stomach cancer, rectal cancer, colon cancer, bladder cancer, lung cancer, breast cancer, liver cancer, pancreatic cancer, gallbladder cancer, bile duct cancer, kidney cancer, cervical cancer, endometrial cancer, ovarian cancer, vaginal cancer, testicular cancer, and prostate cancer.

Examples of the disease in the above-described disease-related antigens may include the above-described cancers, allergic diseases developed in animals including humans (e.g., atopic dermatitis, allergic rhinitis (hay fever), allergic conjunctivitis, allergic gastroenteritis, asthma, food allergy, drug allergy, hives, etc.), and autoimmune diseases (e.g., multiple sclerosis, rheumatoid arthritis, systemic lupus erythematosus, Sjogren's syndrome, systemic scleroderma, ulcerative colitis, Crohn's disease, psoriasis, alopecia areata, type 1 diabetes, Graves' disease, Hashimoto's disease, myasthenia gravis, IgA nephropathy, etc.).

The adjuvant comprised in the vaccine according to the present invention may be comprised in an amount of approximately 0.01% by weight to 99.99% by weight based on 100% by weight of the vaccine. Otherwise, the adjuvant may be comprised in an amount of, for example, approximately 10 weights to 1000 weights based on 1 weight of the antigen.

The vaccine according to the present invention, as a composition, may comprise pharmaceutically acceptable additives, as well as the adjuvant and the antigen. The dosage form of the vaccine composition according to the present invention is not particularly limited, and examples of the dosage form may include a tablet, a capsule, a granule, a power agent, a syrup agent, an inhalant, and a liquid preparation (nasal drops, injection, etc.). These formulations are prepared according to ordinary methods. Beside, such a liquid preparation may be dissolved or suspended in water or another suitable solvent at the time of use. Moreover, such a tablet and a granule may be coated according to publicly known methods. In the caser of an injection, it is prepared by dissolving the compound of the present invention in water. The compound of the present invention may be dissolved in a normal saline or a glucose solution, as necessary, or a buffer or a preservative may be added to the solution.

With regard to the types of the formulation additives used in the vaccine composition according to the present invention, the ratio of the formulation additives to the active ingredient(s), and the like can be selected, as appropriate, by a person skilled in the art, depending on the dosage form. As such formulation additives, inorganic or organic substances, or solid or liquid substances can be used. In general, the formulation additives can be mixed into the vaccine composition in an amount of, for example, 0.1% by weight to 99.9% by weight, 1% by weight to 95.0% by weight, or 1% by weight to 90.0% by weight, based on the weight of the active ingredient(s) (the antigen and/or the adjuvant).

When the vaccine composition according to the present invention is a solid preparation, the active ingredient is mixed with excipient components such as, for example, lactose, starch, crystalline cellulose, calcium lactate and anhydrous silicic acid, to prepare a powder agent. Otherwise, as necessary, binders such as white sugar, hydroxypropyl cellulose and polyvinylpyrrolidone, disintegrators such as carboxymethyl cellulose and carboxymethyl cellulose calcium, and the like are further added to the powder agent, and the obtained mixture is then subjected to wet or dry granulation, so that a granule can be prepared. In addition, in order to produce a tablet, the powder agent and the granule may be subjected to tablet-making, directly or with addition of lubricants such as magnesium stearate and talc. Such a granule or tablet may be coated with enteric base agents such as hydroxypropylmethylcellulose phthalate and a methacrylic acid-methyl methacrylate polymer to prepare an enteric preparation, or such a granule or tablet may be coated with ethyl cellulose, carnauba wax, hydrogenated oil and the like to prepare a sustained release preparation. Further, in order to produce a capsule, the powder agent or the granule is filled into a hard capsule, or the active ingredient is coated with gelatin, directly or after it has been dissolved in glycerin, polyethylene glycol, sesame oil, olive oil and the like, so that a soft capsule can be prepared.

When the vaccine composition according to the present invention is a liquid preparation, the active ingredient is dissolved in distilled water for formulations, as necessary, together with pH adjusters such as hydrochloric acid, sodium hydroxide, lactose, lactic acid, sodium, sodium monohydrogen phosphate and sodium dihydrogen phosphate, and tonicity agents such as sodium chloride and glucose. Then, the obtained solution is subjected to aseptic filtration, and the resulting solution is then filled into an ampoule. Otherwise, mannitol, dextrin, cyclodextrin, gelatin and the like are further added to the resulting solution, followed by vacuum freeze drying, so that a preparation to be dissolved when needed may be prepared. Moreover, lecithin, polysorbate 80, polyoxyethylene hydrogenated castor oil, and the like are added to the active ingredient, and the obtained mixture is then emulsified in water, so that an emulsion for liquid preparations can be prepared.

The disclosures of all publications cited in the present description are incorporated herein by reference in their entirety. In addition, throughout the present description as a whole, when singular terms with the article “a,” “an,” and “the” are used, these terms include not only single items but also multiple items, unless otherwise clearly specified.

Hereinafter, the present invention will be further described in the following examples. However, these examples are only illustrative examples of the embodiments of the present invention, and thus, are not intended to limit the scope of the present invention.

### EXAMPLES

### 1. Experimental Methods and Materials

### 1-1. Cells and Viruses

MDCK (Madin-Darby canine kidney) cells were maintained in MEM medium (Gibco) supplemented with 5% calf serum under conditions of 37° C. and 5% CO2. The MDCK cells were used in a plaque assay for dilution of viruses.

A/California/04/2009 virus (H1N1; MA-CA04) adapted to mice was produced according to the method of the previous report (Sakabe et al., Virus research 158: 124-129, 2009), and was used in infection of mice therewith. A/California/07/2009 virus (H1N1; CA07) was isolated in the early stage of pandemic in 2009, and was provided by National Institute of Infectious Diseases. This virus was used as an antigen in an ELISA assay for determining the virus-specific antibody titer in the serum derived from immunized mice.

### 1-2. Vaccine Antigens and Other Compounds

### 1-2-1. Influenza Vaccine Antigens

Trivalent and quadrivalent split influenza HA vaccines were obtained from DENKA SEIKEN Co., Ltd. (Japan).

The trivalent influenza HA vaccine (used in the 2014-2015 influenza season) comprises A/California/07/2009 (H1N1), A/New York/39/2012 (H3N2), and B/Massachusetts/2/2012 (B/Yamagata lineage). This vaccine was inoculated into mice, and a primary screening was carried out for adjuvant candidates, using the antibody titer specific to the virus in the serum as an indicator.

The quadrivalent split influenza HA vaccine (used in the 2015-2016 influenza season) comprises the HA proteins of A/California/07/2009 (H1N1), A/Switzerland/9715293/2013 (H3N2), B/Phuket/3073/2013 (Yamagata lineage), and B/Texas/2/2013 (Victoria lineage). This vaccine was inoculated into mice, and a secondary screening was carried out for adjuvant candidates, using the protective effect in the mice infected with the virus as an indicator. Moreover, the quadrivalent split influenza HA vaccine (used in the 2016-2017 influenza season) comprises A/California/07/2009 (H1N1), A/Hongkong/4801/2014 (H3N2), B/Phuket/3073/2013 (Yamagata lineage), and B/Texas/2/2013 (Victoria lineage). This vaccine was used in a part of the secondary screening, and was also used to examine the effects thereof against virus replication in the mice infected with the virus.

### 1-2-2. Ebola Virus Antigen

Ebola virus-like particles (VLP) were prepared with reference to the previously reported method (Warfield et al., PLoS One, 10(3): p. e0118881, 2015; Margine et al., J Vis Exp, (81): p. e51112, 2013; Ye et al., Virology, 351: 260-270, 2006; Warfield et al., J Infect Dis, 196 Suppl 2: p. S421-9, 2007). Specifically, the Ebola virus-like particles were prepared using the Bac-to-Bac baculovirus expression system (Invitrogen). A GP gene and a VP40 gene were inserted into the transfer vector pFastBac cleaved with BamHI and Not I. DH10Bac was transformed with the recombinants pFastBac-GP and pFastBac-VP40, so that individual recombinant bacmids were produced. The recombinant bacmid, into which the Ebola GP gene or VP40 gene had been inserted, was purified, and using Cellfectin II reagent (Invitrogen), the obtained recombinant bacmid was introduced into sf90 cells. Six days after the transformation, a recombinant baculovirus rBV-GP and a recombinant baculovirus rBV-VP40 were recovered as P1 viruses from the medium. A virus stock to be used was prepared by amplifying the P1 virus twice in sf9 cells.

High Five cells in a suspending culture were co-infected with rBV-GP and rBV-VP40, and the resulting High Five cells were then cultured in a magnetic culture vessel (250 ml of culture medium/1 L volume) at 28° C. Sixty hours after the infection, the medium was recovered and was then centrifuged at 3,500 rpm at 4° C. for 15 minutes. The obtained supernatant was concentrated, and was then layered on 25% sucrose, followed by centrifugation at 28,000 rpm at 4° C. for 1.5 hours. The obtained precipitate was suspended in PBS. The purified VLP was dispensed and was then preserved at −80° C. before the use thereof. The total protein concentration was determined using BCA Protein Assay kit (Thermo Fisher Scientific).

### 1-2-3. Adjuvant Candidate Compounds

2% Aluminum hydroxide gel adjuvant (Alum), Alhydrogel (registered trademark), was purchased from InvivoGen, and was used as a positive control (antigen:alum v/v)=1:1). Food additive-derived compounds and injection additive-derived compounds were each purchased from the companies shown in Table 1 and Table 5. These compounds were each diluted with a phosphate buffered saline (PBS) (calcium- and magnesium-free) to a concentration of 10 mg/ml or 10 μl/ml, and were then subjected to an ultrasonic treatment in a water bath for 15 minutes at room temperature. The compound stocks were preserved at −20° C. before the use thereof. After thawing, the stocks were each subjected to an ultrasonic treatment for 5 minutes before being mixed with a vaccine antigen.

The applied dose of an adjuvant candidate compound was set to be 100 μg/dose (provided that the dose of saponin was set to be 10 μg/dose).

### 1-3. Inoculation of Vaccines

### 1-3-1. Influenza Vaccines

### (1) Effects as Intramuscular Injection Vaccine Adjuvants

Five-week-old female BALB/c mice were purchased from Japan SLC, Inc. The mice were acclimated for 1 week, and thereafter, only the vaccine, or the vaccine+the adjuvant candidate compound in an amount equal to or smaller than the optimal dose (i.e., the vaccine for the 2014-2015 season: 0.01 μg/dose; the vaccine for the 2015-2016 season: 0.003 μg/dose; and the vaccine for the 2016-2017 season: 0.001 μg/dose) was inoculated into the femur of each mouse via intramuscular injection. Fourteen days after the first inoculation, a second inoculation was carried out. Fourteen days after the second inoculation, blood was sampled from the facial vein, using Goldenrod Animal Lancet (5 mm), and the serum was then prepared for the measurement of a virus-specific antibody titer, which was then subjected to a primary screening. In a secondary screening, vaccination was carried out in the same manner as the primary screening, and 21 days after the second vaccination, the mice were infected with the MA-CA04 virus at a dose of 10 MLD₅₀ (which was the amount by which 50% of the infected mice died). For 14 days after the viral infection, the body weight and survival of the mice were monitored every day. The mice whose body weight was reduced by more than 25% of the body weight before the infection were euthanized. Three or four mice per group were used in the screening.

The effects of the adjuvant candidate compounds against the virus replication in the respiratory tract of the mice infected with the influenza virus were examined as follows.

Various types of antigens (only PBS, only the adjuvant candidate compound, only the vaccine, the vaccine+the adjuvant candidate compound, and the vaccine+Alum) were each inoculated at a dose of 100 μl into the femur of 6-week-old BALB/c mice via intramuscular injection. Three weeks after vaccine booster, the mice were infected with the MA-CA04 virus at a dose of 10 MLD₅₀ twice with an interval of 2 weeks. Three days and six days after the infection, the nasal concha and the lung were excised from the mice (n=3), and were then homogenized. Thereafter, using MDCK cells, a plaque assay was carried out.

### (2) Effects of Nasal Vaccine Adjuvants

With regard to the additives that had exhibited the effects of adjuvants when they had been applied by intramuscular injection, the effects of the additives as nasal vaccine adjuvants were examined.

Before the mixing of a vaccine antigen, a suspension or a solution of the adjuvant candidate compounds was melted in a water bath, and was then subjected to an ultrasonic treatment (in which a treatment for 20 seconds and a standing for 30 seconds were repeated for 10 minutes). While a mixed solution of a vaccine antigen and an adjuvant candidate compound was shaken, it was incubated at 4° C. for 3 hours. Thereafter, the mice were immunized with the prepared vaccine mixed solution three times (Day 0, Day 14, and Day 28), and forty-two days after the immunization, blood, a nasal lavage fluid, and a bronchoalveolar lavage fluid (BALF) were collected, followed by measuring the antibody titer. For the immunization, 5 μl for a nostril, and a total of 10 μl (100 μg/dose) was administered to each mouse. However, the doses of saponin and poly(I:C) (positive control) were each set to be 10 μg/dose. 21 days after the second vaccination, the mice were infected with the MA-CA04 virus at a dose of 10 MLD₅₀ (which was the amount by which 50% of the infected mice died). For 14 days after the viral infection, the body weight and survival of the mice were monitored every day. The mice whose body weight was reduced by more than 25% of the body weight before the infection were euthanized. Three or four mice per group were used in the screening.

### 1-3-2. Ebola Vaccines

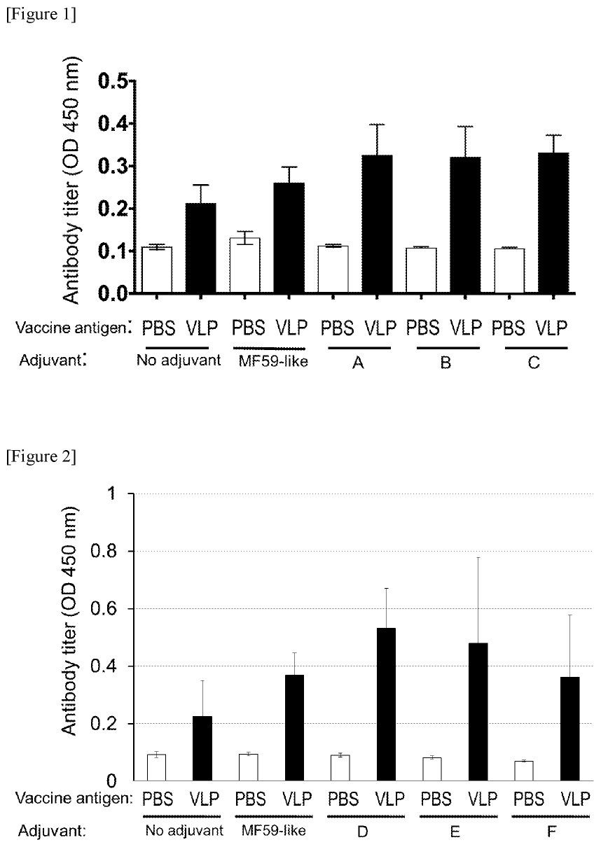

### (1) Concerning FIG. 1 and FIG. 2

Five-week-old female BALB/c mice were purchased from Japan SLC, Inc. The mice were acclimated for 1 week, and thereafter, only the vaccine, or the vaccine+the adjuvant candidate compound, was inoculated into the femur of each mouse via intramuscular injection. As an Ebola vaccine antigen, Ebola virus-like particles (virus-like particles: VLP) were inoculated at 10 μg/dose into the mice.

When calcium glycerophosphate hydrate, sodium chondroitin sulfate, and riboflavin were used as additive compounds serving as adjuvants, the second vaccination was carried out 12 days after the first vaccination. Twelve days after the second vaccination, using Goldenrod Animal Lancet (5 mm), blood was sampled from the facial vein, and serum was then prepared for the measurement of a virus-specific antibody titer. Thereafter, the antibody titer against the Ebola GP protein was measured.

Moreover, when EMANON CH-60K, hydroxypropyl cellulose, and polyoxyethylene polyoxypropylene glycol)(160E.O.) (30P.O.) were used as additive compounds serving as adjuvants, the antibody titer against the Ebola GP protein was measured 2 weeks after the first vaccination.

### (2) Concerning Table 11

The antigen (1 μg of Ebola virus-like particles/dose) was inoculated at a dose of 100 μl into 6-week-old BALB/c mice twice with an interval of 2 weeks via intramuscular injection. Four mice were allocated to each group (a group inoculated with only the Ebola virus VLP, a group inoculated with the Ebola virus VLP+an adjuvant candidate compound, and a group inoculated with the Ebola virus VLP+AddVax (a positive control adjuvant). In order to measure an IgG antibody titer specific to the Ebola GP protein, blood was sampled two weeks after the second immunization, and serum was then prepared therefrom.

### 1-4. Measurement of Antibody Titer of Ebola GP Protein-Specific Antibody

### (1) Concerning FIG. 1 and FIG. 2

The antibody titer in the serum was measured by applying a modified method of ELISA, as described in the previous report (Uraki et al., Vaccine 32: 5295-5300, 2014). The soluble GP protein (the GP mutant T42V/T230V GP1-632Δmuc) used in the ELISA was prepared from GP coded DNA synthesized by overlapping PCR according to the method of the previous report (Lee et al., Nature, 454: 177-182, 2008).

A 96-well ELISA plate (IWAKI) was coated with a purified CA07 virus solution (6 μg/ml) or an Ebola GP protein (5 μg/ml) at 4° C. overnight (50 μl/well). Thereafter, the plate was blocked with 200 μl of a 20% Blocking One (Nacalai) aqueous solution at room temperature for 1 hour. After completion of the blocking, the plate was washed with PBS (PBS-T) containing 0.05% Tween-20 once, and thereafter, a two-fold serially diluted serum sample was added to the plate, followed by incubation at room temperature for 1 hour. The bound IgG was detected using a F(ab′)2 fragment of a peroxidase-labeled goat anti-mouse IgG (γ) antibody (Kirkegaard & Perry Laboratories, Inc.). The plate was washed with PBS-T, and 100 μl of a 2,2′-azino-bis(3-ethylbenzothiazoline-6-sulphonic acid) substrate solution was then added to each well, so that a coloration reaction was initiated. After completion of the reaction, OD was measured at a wavelength of 405 nm.

### (2) Concerning Table 11

Using a purified GP mutant as a coating antigen, an Ebola GP protein-specific antibody titer (IgG antibody titer) was determined according to an ELISA method. OD was measured at a wavelength of 405 nm. The antibody titer was defined as a reciprocal of the serum dilution at a reaction termination point of OD 405>0.1. The value was indicated as a mean value of the antibody titers of 4 mice in each group.

### 1-5. Statistical Processing

Significant differences in the antibody titers and the survival rates of the virus-infected mice were obtained according to one-way analysis of variance (one-way ANOVA) using GraphPad Prism 6 software. A significant difference in the virus titers was evaluated according to an unpaired two-tailed t-test. P value <0.05 was defined to be a statistical significant difference.

### 1-6. Ethical Treatment

All experiments involving the use of mice were carried out in accordance with Animal Feeding Regulation, University of Tokyo, and the guidelines for appropriate implementation of animal experiments by the Science Council of Japan. Also, the animal experiments were carried out with the approval of the Animal Care and Use Committee, the Institute of Medical Science, the University of Tokyo.

### 2. Results

### 2-1. Primary Screening

In order to seek novel adjuvants, screening was carried out on the compounds derived from the food additives and injection additives approved in Japan, using mice. From a list of food additives and injection additives, 145 compounds derived from food additives and 51 compounds derived from injection additives, from which enzymes, salts, simple substances, proteins, biological low-molecular-weight compounds, waxes, hydrocarbons and soil are excluded, were selected as targets of the screening. An HA vaccine, to which commercially available alum was added, was used as a positive control.

Two weeks after vaccine booster, an influenza virus-specific antibody titer was measured in each of serum samples collected from the vaccinated mice. No antibodies against the CA07 virus were detected in mice inoculated with only PBS and with only the compound. In addition, in mouse serums inoculated with only the HA vaccine, no virus-specific antibodies could be detected, or even if such antibody could be detected, it was at a low level (antibody dilution range <10 to 320). Thus, in a primary screening, a compound capable of inducing a mean antibody titer >320 when it was inoculated together with the HA vaccine was defined as a hit compound, and 59 compounds were selected from the food additive-derived compounds, which were then subjected to a secondary screening. Regarding the injection additive-derived compounds, all of the 51 compounds were subjected to the secondary screening.

### 2-2. Secondary Screening (Identification of Adjuvant Candidate Compounds)

Subsequently, the compounds selected in the primary screening were subjected to a secondary screening, using the protective effect of the vaccine in mice infected with a fatal dose of virus as an indicator. In the secondary screening, a quadrivalent split influenza HA vaccine (used in the 2015-2016 season, or in the 2016-2017 season) was used as an HA vaccine. The vaccine used in the 2015-2016 season was inoculated at 0.003 μg/dose, whereas the vaccine used in the 2016-2017 season was inoculated at 0.001 μg/dose. Three weeks after the second vaccination, the mice were infected with the MA-CA04 virus at a dose of 10 MLD₅₀, and the body weight and survival of the mice were then monitored for 14 days. In the secondary screening, compounds inoculated into mouse groups each exhibiting a survival rate equal to or higher than the survival rate of the mouse group inoculated with the alum adjuvant (positive control) were selected. Forty-one compounds derived from food additives identified by the secondary screening are shown in Table 1, whereas 21 compounds derived from injection additives identified by the secondary screening are shown in Table 5. Moreover, the antibody titers of the 41 compounds derived from food additives in the primary screening are shown in Table 2, whereas the antibody titers of the 41 compounds derived from food additives and the 21 compounds derived from injection additives in the secondary screening are shown in Table 3 and Table 6, respectively. Furthermore, the protective effects against viral infection of the 41 compounds derived from food additives and the 21 compounds derived from injection additives in the secondary screening are shown in Table 4 and Table 7, respectively.

TABLE 1

| Compounds | Function in Food | CAS No. | Company | Remarks |
|---|---|---|---|---|
| Abietic acid | Preservative, acidity regulator | 514-10-3 | Sigma | a |
| Brilliant Blue FCF | Color | 3844-45-9 | Wako | a |
| Carminic acid | Color | 1260-17-9 | Wako | a |
| (+/−)-Citronellol | Flavor | 106-22-9 | Wako | a |
| (R)-(+)-Citronellal | Flavor | 2385-77-5 | Sigma | a |
| Crocin | Color | 42553-65-1 | Sigma | a |
| Fast Green FCF | Color | 2353-45-9 | Sigma | a |
| Geranyl Formate | Flavor | 105-86-2 | Wako | a |
| Hydroxycitronellal | Flavor | 107-75-5 | Wako | a |
| Indigo Carmine | Color | 860-22-0 | Wako | a |
| Iron (II) Gluconate n-hydrrate | Color retention agent | 299-29-6 | Wako | a |
| Isoeugenol | Flavor | 97-54-1 | Sigma | a |
| Methyl Anthranilate | Flavor enhancer | 134-20-3 | Wako | a |
| Naringin | Antioxidant | 10236-47-2 | Sigma | a |
| Neotame | Sweetener | 165450-17-9 | Sigma | a |
| Norbixin | Color | 542-40-5 | CAROTE NATURE | a |
| Terpineol | Flavor | 8006-39-1 | Wako | a |
| γ-Undecalactone | Flavor | 104-67-6 | Sigma | a |
| Calcium glycerophosphate hydrate | Thickener, gelling agent, stabilizer | 27214-00-2 | Sigma | b |
| Calcium sorbate | Preservative | 7492-55-9 | Fluorochem | b |
| β-Carotene | Color | 7235-40-7 | Sigma | b |
| Chondroitin sulfate sodium salt | Emulsifier, stabilizer | 9082/7/9 | ChromaDex | b |
| β-D-Glucan | Antioxidant | 9041-22-9 | Sigma | b |
| Glycyrrhizic acid ammonium salt | Sweetener | 53956-04-0 | Sigma | b |
| Hesperidin | Antioxidant, nutrient supplement | 520-26-3 | Sigma | b |
| Isoquinoline | Color | 119-65-3 | Wako | b |
| Pectin | Vegetable gum, emulsifier | 9000-69-5 | Sigma | b |
| Polysorbate 20 | Emulsifier | 9005-64-5 | Sigma | b |
| Polysorbate 60 | Emulsifier | 9005-67-8 | Sigma | b |
| Polysorbate 80 | Emulsifier | 9005-65-6 | Sigma | b |
| Rutin | Color, antioxidant, nutrient supplement | 153-18-4 | Wako | b |
| Rutin hydrate | Color, antioxidant, nutrient supplement | 207671-50-9 | Sigma | b |
| Theobromine | Flavor | 83-67-0 | Sigma | b |
| β-Cyclodextrin | Emulsifier | 7585-39-9 | Sigma | c |
| Poly-L-γ-glutamic acid sodium salt | Flavor enhancer | 26247-79-0 | Sigma | c |
| Polyvinylpyrrolidone (MW: 3,600,000) | Emulsifier, stabilizer | 9003-39-8 | Sigma | c |
| Pullulan | Thickener, gelling agent | 9057/2/7 | Sigma | c |
| Riboflavin | Color | 83-88-5 | Wako | c |
| Saponin | Foaming agent | 8047-15-2 | Sigma | c |
| Sepiolite | Acidity regulator, anticaking agent | 63800-37-3 | Sigma | c |
| Sodium Alginate 80-120 | Thickener, stabilizer | 194-13321 | Wako | c |

Note)
a: Compounds not reported as adjuvants
b: Compounds that have not been reported as influenza vaccine adjuvants but have been reported as other virus vaccine adjuvants
c: Compounds reported as influenza vaccine adjuvants

TABLE 2

| | Antibody titers in primary screening | | | Relative ratio of |
|---|---|---|---|---|
| | Only | Vaccine + | Vaccine + | antibody titer to alum |
| Compounds | vaccine | compound | alum | (compound/alum) |
| Abietic acid | <10 | 533.33 | 233.33 | 2.29 |
| Brilliant Blue FCF | <10 | 1333.33 | 1920.00 | 0.69 |
| Carminic acid | <10 | 746.67 | 1920.00 | 0.39 |
| (+/−)-Citronellol | <10 | 960.00 | 1706.67 | 0.56 |
| (R)-(+)-Citronellal | <10 | 333.33 | 1706.67 | 0.20 |
| Crocin | <10 | 1066.67 | 1706.67 | 0.63 |
| Fast Green FCF | <10 | 2133.33 | 2133.33 | 1.00 |
| Geranyl Formate | <10 | 2560.00 | 853.33 | 3.00 |
| Hydroxycitronellal | <10 | 2773.33 | 853.33 | 3.25 |
| Indigo Carmine | <10 | 1280.00 | 853.33 | 1.50 |
| Iron (II) Gluconate n-hydrrate | <10 | 853.33 | 1120.00 | 0.76 |
| Isoeugenol | <10 | 2986.67 | 853.33 | 3.50 |
| Methyl Anthranilate | <10 | 693.33 | 853.33 | 0.81 |
| Naringin | 320 | 2133.33 | 5120.00 | 0.42 |
| Neotame | 40 | 2560.00 | 1280.00 | 2.00 |
| Norbixin | 113.33 | 7680.00 | 960.00 | 8.00 |
| Terpineol | <10 | 853.33 | 1280.00 | 0.67 |
| γ-Undecalactone | 113.33 | 960.00 | 960.00 | 1.00 |
| Calcium glycerophosphate hydrate | <10 | 1920.00 | 1120.00 | 1.71 |
| Calcium sorbate | <10 | 2133.33 | 1920.00 | 1.11 |
| β-Carotene | <10 | 960.00 | 1706.67 | 0.56 |
| Chondroitin sulfate sodium salt | <10 | 1493.33 | 1120.00 | 1.33 |
| β-D-Glucan | <10 | 2560.00 | 1706.67 | 1.50 |
| Glycyrrhizic acid ammonium salt | <10 | 5973.33 | 3413.33 | 1.75 |
| Hesperidin | <10 | 3840.00 | 3413.33 | 1.13 |
| Isoquinoline | <10 | 1493.33 | 853.33 | 1.75 |
| Pectin | 320 | 640.00 | 5120.00 | 0.13 |
| Polysorbate 20 | 40 | 1920.00 | 1280.00 | 1.50 |
| Polysorbate 60 | 40 | 2560.00 | 1280.00 | 2.00 |
| Polysorbate 80 | 40 | 3416.33 | 1280.00 | 2.67 |
| Rutin | 320 | 2560.00 | 5120.00 | 0.50 |
| Rutin hydrate | 320 | 1280.00 | 5120.00 | 0.25 |
| Theobromine | <10 | 1333.33 | 1706.67 | 0.78 |
| β-Cyclodextrin | <10 | 373.33 | 1706.67 | 0.22 |
| Poly-L-γ-glutamic acid sodium salt | 320 | 2560.00 | 5120.00 | 0.50 |
| Polyvinylpyrrolidone (MW: 3,600,000) | <10 | 506.67 | 480.00 | 1.06 |
| Pullulan | <10 | 960.00 | 1706.67 | 0.56 |
| Riboflavin | <10 | 2560.00 | 2133.33 | 1.20 |
| Saponin | <10 | 4266.67 | 480.00 | 8.89 |
| Sepiolite | 320 | 2986.67 | 5120.00 | 0.58 |
| Sodium Alginate 80-120 | <10 | 853.33 | 1706.67 | 0.50 |

TABLE 3

| | Antibody titers in secondary screening | | | Relative ratio of |
|---|---|---|---|---|
| | Only | Vaccine + | Vaccine + | antibody titer to alum |
| Compounds | vaccine | compound | alum | (compound/alum) |
| Abietic acid | 60 | 1280 | 560 | 2.29 |
| Brilliant Blue FCF | <10 | 990 | 600 | 1.65 |
| Carminic acid | 110 | 960 | 1440 | 0.67 |
| (+/−)-Citronellol | <10 | 260 | 720 | 0.36 |
| (R)-(+)-Citronellal | <10 | 35 | 1600 | 0.02 |
| Crocin | <10 | 480 | 720 | 0.67 |
| Fast Green FCF | <10 | 440 | 600 | 0.73 |
| Geranyl Formate | <10 | 960 | 1600 | 0.60 |
| Hydroxycitronellal | 110 | 1280 | 1440 | 0.89 |
| Indigo Carmine | <10 | 485 | 600 | 0.81 |
| Iron (II) Gluconate n-hydrrate | <10 | 3040 | 1600 | 1.90 |
| Isoeugenol | <10 | 140 | 720 | 0.19 |
| Methyl Anthranilate | 380 | 2240 | 2560 | 0.88 |
| Naringin | 380 | 2240 | 840 | 2.67 |
| Neotame | <10 | 720 | 720 | 1.00 |
| Norbixin | 110 | 2880 | 560 | 5.14 |
| Terpineol | 380 | 2880 | 2560 | 1.13 |
| γ-Undecalactone | 380 | 1920 | 840 | 2.29 |
| Calcium glycerophosphate hydrate | <10 | 4800 | 1440 | 3.33 |
| Calcium sorbate | <10 | 3520 | 1440 | 2.44 |
| β-Carotene | 60 | 1120 | 560 | 2.00 |
| Chondroitin sulfate sodium salt | <10 | 2880 | 1440 | 2.00 |
| β-D-Glucan | 60 | 2560 | 560 | 4.57 |
| Glycyrrhizic acid ammonium salt | 110 | 2560 | 1440 | 1.78 |
| Hesperidin | 60 | 2240 | 560 | 4.00 |
| Isoquinoline | 110 | 1200 | 1440 | 0.83 |
| Pectin | <10 | 320 | 600 | 0.53 |
| Polysorbate 20 | 205 | 5760 | 2240 | 2.57 |
| Polysorbate 60 | 205 | 2560 | 2240 | 1.14 |
| Polysorbate 80 | <10 | 4160 | 1600 | 2.60 |
| Rutin | <10 | 1320 | 1440 | 0.92 |
| Rutin hydrate | <10 | 720 | 720 | 1.00 |
| Theobromine | 60 | 1440 | 560 | 2.57 |
| β-Cyclodextrin | <10 | 1440 | 1600 | 0.90 |
| Poly-L-γ-glutamic acid sodium salt | <10 | 60 | 1600 | 0.04 |
| Polyvinylpyrrolidone (MW: 3,600,000) | <10 | 2720 | 1600 | 1.70 |
| Pullulan | 380 | 720 | 840 | 0.86 |
| Riboflavin | <10 | 2240 | 1440 | 1.56 |
| Saponin | 205 | 12160 | 2240 | 5.43 |
| Sepiolite | 300 | 2080 | 2560 | 0.81 |
| Sodium Alginate 80-120 | 205 | 1280 | 2240 | 0.57 |

TABLE 4

| | Protective effects (Number of surviving/Total number) | | | | |
|---|---|---|---|---|---|
| | Only | Only | Vaccine + | Vaccine + | Protective effect- |
| Compounds | compound | vaccine | compound | alum | enhancing rate (%) |
| Abietic acid | 0/4 | 1/4 | 3/4 | 3/4 | 50 |
| Brilliant Blue FCF | 0/4 | 0/4 | 3/4 | 3/4 | 75 |
| Carminic acid | 0/4 | 1/4 | 4/4 | 3/4 | 75 |
| (+/−)-Citronellol | 0/4 | 0/4 | 3/4 | 3/4 | 75 |
| (R)-(+)-Citronellal | 0/4 | 0/4 | 2/4 | 2/4 | 50 |
| Crocin | 0/4 | 0/4 | 4/4 | 3/4 | 100 |
| Fast Green FCF | 0/4 | 0/4 | 2/4 | 3/4 | 50 |
| Geranyl Formate | 0/4 | 0/4 | 2/4 | 3/4 | 50 |
| Hydroxycitronellal | 0/4 | 1/4 | 4/4 | 3/4 | 75 |
| Indigo Carmine | 0/4 | 0/4 | 2/4 | 3/4 | 50 |
| Iron (II) Gluconate n-hydrrate | 0/4 | 0/4 | 3/4 | 2/4 | 75 |
| Isoeugenol | 0/4 | 0/4 | 2/4 | 3/4 | 50 |
| Methyl Anthranilate | 0/4 | 1/4 | 4/4 | 3/4 | 75 |
| Naringin | 0/4 | 1/4 | 3/4 | 3/4 | 50 |
| Neotame | 0/4 | 0/4 | 4/4 | 3/4 | 100 |
| Norbixin | 0/4 | 1/4 | 4/4 | 3/4 | 75 |
| Terpineol | 0/4 | 1/4 | 4/4 | 3/4 | 75 |
| γ-Undecalactone | 0/4 | 1/4 | 4/4 | 3/4 | 75 |
| Calcium glycerophosphate hydrate | 0/4 | 0/4 | 3/4 | 2/4 | 75 |
| Calcium sorbate | 0/4 | 0/4 | 4/4 | 2/4 | 100 |
| β-Carotene | 0/4 | 1/4 | 4/4 | 3/4 | 75 |
| Chondroitin sulfate sodium salt | 0/4 | 0/4 | 4/4 | 2/4 | 100 |
| β-D-Glucan | 0/4 | 1/4 | 4/4 | 3/4 | 75 |
| Glycyrrhizic acid ammonium salt | 0/4 | 1/4 | 4/4 | 3/4 | 75 |
| Hesperidin | 0/4 | 1/4 | 4/4 | 3/4 | 75 |
| Isoquinoline | 0/4 | 1/4 | 3/4 | 3/4 | 50 |
| Pectin | 0/4 | 0/4 | 2/4 | 3/4 | 50 |
| Polysorbate 20 | 0/4 | 1/4 | 4/4 | 2/4 | 75 |
| Polysorbate 60 | 0/4 | 1/4 | 2/4 | 2/4 | 25* |
| Polysorbate 80 | 0/4 | 0/4 | 2/3** | 2/4 | 66.7 |
| Rutin | 0/4 | 0/4 | 2/4 | 2/4 | 50 |
| Rutin hydrate | 0/4 | 0/4 | 4/4 | 3/4 | 100 |
| Theobromine | 0/4 | 1/4 | 4/4 | 3/4 | 75 |
| β-Cyclodextrin | 0/4 | 0/4 | 4/4 | 2/4 | 100 |
| Poly-L-γ-glutamic acid sodium salt | 0/4 | 0/4 | 2/4 | 2/4 | 50 |
| Polyvinylpyrrolidone (MW: 3,600,000) | 0/4 | 0/4 | 3/4 | 2/4 | 75 |
| Pullulan | 0/4 | 1/4 | 3/4 | 3/4 | 50 |
| Riboflavin | 0/4 | 0/4 | 4/4 | 2/4 | 100 |
| Saponin | 0/4 | 1/4 | 4/4 | 2/4 | 75 |
| Sepiolite | 0/4 | 1/4 | 3/4 | 3/4 | 50 |
| Sodium Alginate 80-120 | 0/4 | 1/4 | 4/4 | 2/4 | 75 |

*Since Polysorbate 60 had protective effects equivalent to those of alum in the same batch, it was identified as a hit compound in the secondary screening.
**One out of four mice inoculated with vaccine + Polysorbate 80 died after blood sampling.

Among the 41 compounds derived from food additives identified by the secondary screening, 18 compounds were novel adjuvant candidate compounds, 15 compounds had been reported to be virus vaccine adjuvant candidates against viruses other than influenza virus, and 8 compounds had bene reported to be influenza virus vaccine adjuvant candidates (Table 1). Among the 18 compounds as novel adjuvant candidates, 8 compounds, namely, carminic acid, crocin, hydroxycitronellal, methyl anthranilate, neotame, norbixin, terpineol, and γ-undecalactone were inoculated together with the vaccine into 4 mice. As a result, all of the 4 mice survived, and thus, these 8 compounds exhibited excellent protective effects against the attack by the MA-CA04 virus (Table 4). Moreover, neotame, norbixin, and γ-undecalactone induced virus-specific antibody titers that were equal to or higher than the alum adjuvant did (Table 2 and Table 3). In particular, norbixin induced the highest antibody titer among the 18 hit compounds (wherein the relative ratio of the antibody titer of norbixin to that of the alum adjuvant was 5.14; Table 3). When compared with the mouse groups inoculated only with the HA vaccine, pathological condition and fatality rate (evaluated with a body weight change and a survival rate) were significantly reduced in the mouse groups inoculated with the HA vaccine+norbixin.

TABLE 5

| Compounds | CAS No. | Company | Remarks |
|---|---|---|---|
| Ammonium acetate | 631-61-8 | Sigma | a |
| EMANON CH-25 | 61788-85-0 | Kao Chemicals | a |
| EMANON CH-40 | 61788-85-0 | Kao Chemicals | a |
| EMANON CH-60K | 61788-85-0 | Kao Chemicals | a |
| Ethanol | 64-17-5 | Wako | a |
| Sodium D-Gluconate | 527-07-1 | Sigma | a |
| Hydroxypropyl cellulose | 9004-64-2 | Wako | a |
| Potassium chloride | 7447-40-7 | Sigma | a |
| Sodium acetate | 127-09-3 | Wako | a |
| Sodium benzoate | 532-32-1 | Sigma | a |
| Sodium hydrogen sulfite | 7631-90-5 | Sigma | a |
| Sodium bromide | 7647-15-6 | Sigma | a |
| Sodium sulfite | 7757-83-7 | Sigma | a |
| Sodium thiosulfate | 10102-17-7 | TCI | a |
| 1,1,1-Trichloro-2-methyl-2- | 6001-64-5 | Wako | a |
| propanol hemihydrate | | | |
| Xylitol | 87-99-0 | Sigma | a |
| Dextran 40 | 9004-54-0 | TCI | b |
| Gum Arabic | 9000-01-5 | Sigma | b |
| Polyethylene glycol 4,000 | 25322-68-3 | TCI | b |
| Polyoxyethylene | 25322-68-3 | Wako | b |
| polyoxypropylene glycol | | | |
| (160E.O.) (30P.O.) | | | |
| RHEODOL AO-15V | 8007-43-0 | Kao Chemicals | b |

Note)
a: Compounds not reported as adjuvants
b: Compounds that have not been reported as influenza vaccine adjuvants but have been reported as other virus vaccine adjuvants
c: Compounds reported as influenza vacine adjuvants

TABLE 6

| | Mean value of antibody titers | | | | Relative ratio of |
|---|---|---|---|---|---|
| | Only | Only | Vaccine + | Vaccine + | antibody titer to alum |
| Compounds | compound | vaccine | compound | alum | (Compound/alum) |
| Ammonium acetate | <10 | <10 | 1200.00 | 1120.00 | 1.07 |
| EMANON CH-25 | <10 | <10 | 3520.00 | 2400.00 | 1.47 |
| EMANON CH-40 | <10 | <10 | 1760.00 | 2400.00 | 0.73 |
| EMANON CH-60K | <10 | <10 | 3520.00 | 2400.00 | 1.47 |
| Ethanol | <10 | <10 | 400.00 | 720.00 | 0.56 |
| Sodium D-Gluconate | <10 | <10 | 1600.00 | 1600.00 | 1.00 |
| Hydroxypropyl cellulose | <10 | <10 | 5440.00 | 2080.00 | 2.62 |
| Potassium chloride | <10 | <10 | 2920.00 | 1600.00 | 1.83 |
| Sodium acetate | <10 | <10 | 740.00 | 1120.00 | 0.66 |
| Sodium benzoate | <10 | <10 | 1600.00 | 1320.00 | 1.21 |
| Sodium hydrogen sulfite | <10 | <10 | 730.00 | 1320.00 | 0.55 |
| Sodium bromide | <10 | <10 | 970.00 | 1120.00 | 0.87 |
| Sodium sulfite | <10 | <10 | 2080.00 | 1600.00 | 1.30 |
| Sodium thiosulfate | <10 | <12.5 | 1320.00 | 1320.00 | 1.00 |
| 1,1,1-Trichloro-2-methyl-2-propanol | <10 | <10 | 1680.00 | 1120.00 | 1.50 |
| hemihydrate | | | | | |
| Xylitol | <10 | <10 | 560.00 | 1600.00 | 0.35 |
| Dextran 40 | <10 | <12.5 | 1920.00 | 1320.00 | 1.45 |
| Gum Arabic | <10 | <10 | 1360.00 | 1600.00 | 0.85 |
| Polyethylene glycol 4,000 | <10 | <10 | 1066.67 | 1600.00 | 0.67 |
| Polyoxyethylene polyoxypropylene | <10 | <10 | 2560.00 | 2400.00 | 1.07 |
| glycol (160E.O.) (30P.O.) | | | | | |
| RHEODOL AO-15V | <10 | <12.5 | 3200.00 | 1320.00 | 2.42 |

TABLE 7

| | Protective effects (Number of surviving/Total number) | | | | |
|---|---|---|---|---|---|
| | Only | Only | Vaccine + | Vaccine + | Protective effect- |
| Compounds | compound | vaccine | compound | alum | enhancing rate (%) |
| Ammonium acetate | 0/4 | 0/4 | 2/4 | 2/4 | 50 |
| EMANON CH-25 | 0/4 | 0/4 | 4/4 | 2/4 | 100 |
| EMANON CH-40 | 0/4 | 0/4 | 3/4 | 2/4 | 75 |
| EMANON CH-60K | 0/4 | 0/4 | 4/4 | 2/4 | 100 |
| Ethanol | 0/4 | 0/4 | 3/4 | 3/4 | 75 |
| Sodium D-Gluconate | 0/4 | 0/4 | 3/4 | 2/4 | 75 |
| Hydroxypropyl cellulose | 0/4 | 0/4 | 4/4 | 2/4 | 100 |
| Potassium chloride | 0/4 | 0/4 | 2/4 | 2/4 | 50 |
| Sodium acetate | 0/4 | 0/4 | 2/4 | 2/4 | 50 |
| Sodium benzoate | 0/4 | 0/4 | 4/4 | 2/4 | 100 |
| Sodium hydrogen sulfite | 0/4 | 0/4 | 3/4 | 2/4 | 75 |
| Sodium bromide | 0/4 | 0/4 | 3/4 | 2/4 | 75 |
| Sodium sulfite | 0/4 | 0/4 | 4/4 | 2/4 | 100 |
| Sodium thiosulfate | 0/4 | 0/4 | 3/4 | 3/4 | 75 |
| 1,1,1-Trichloro-2-methyl-2-propanol | 0/4 | 0/4 | 3/4 | 2/4 | 75 |
| hemihydrate | | | | | |
| Xylitol | 0/4 | 0/4 | 2/4 | 2/4 | 50 |
| Dextran 40 | 0/4 | 0/4 | 3/4 | 3/4 | 75 |
| Gum Arabic | 0/4 | 0/4 | 3/4 | 2/4 | 75 |
| Polyethylene glycol 4,000 | 0/4 | 0/4 | 2/4 | 2/4 | 50 |
| Polyoxyethylene polyoxypropylene | 0/4 | 0/4 | 4/4 | 2/4 | 100 |
| glycol (160E.O.) (30P.O.) | | | | | |
| RHEODOL AO-15V | 0/4 | 0/4 | 3/4 | 3/4 | 75 |

Among the 21 compounds derived from injection additives identified by the secondary screening, 16 compounds were novel adjuvant candidate compounds, and 5 compounds had been reported to be virus vaccine adjuvant candidates against viruses other than influenza virus (Table 5). Among the 16 compounds as novel adjuvant candidates, 5 compounds, namely, EMANON CH-25, EMANON CH-60K, hydroxypropyl cellulose, sodium benzoate, and sodium sulfite were inoculated together with the vaccine into 4 mice. As a result, all of the 4 mice survived, and thus, these 5 compounds exhibited excellent protective effects against the attack by the MA-CA04 virus (Table 7). Moreover, these 5 compounds induced virus-specific antibody titers that were equal to or higher than the alum adjuvant did (Table 6). In particular, hydroxypropyl cellulose induced the highest antibody titer among the 21 hit compounds.

### 2-3. Effects of Adjuvant Candidate Compounds Against Virus Replication in Mouse Respiratory Tract

Neotame, norbixin, and γ-undecalactone (derived from food additives), and EMANON CH-25, EMANON CH-60K, hydroxypropyl cellulose, sodium benzoate, and sodium sulfite (derived from injection additives), had exhibited excellent protective effects against viral infection. The influence of these compounds on virus replication was examined.

Mice inoculated with the HA vaccine and each candidate compound were infected with the MA-CA04 virus at a dose of 10 MLD₅₀, three weeks after the second vaccination. On Day 3 and Day 6 after the viral infection, organ samples (nasal concha and lung) were collected from the euthanized mice. Three days after the infection, the virus with a high titer was recovered from both the nasal concha and the lung of all groups (Table 8: derived from food additives; Table 9: derived from injection additives). In contrast, Six days after the infection, the virus titers in the nasal concha and the lung tended to be decreased in the groups inoculated with the HA vaccine and the adjuvant candidate compound, in comparison to the groups inoculated with only the HA vaccine, and in particular, no virus titers were detected in some mice inoculated with neotame (Table 8), EMANON CH-25, EMANON CH-60K, hydroxypropyl cellulose, and sodium benzoate (Table 9). These results suggested that several adjuvant candidates such as neotame, EMANON CH-25, EMANON CH-60K, hydroxypropyl cellulose, and sodium benzoate have the function of promptly eliminating viruses from the body of the mice.

TABLE 8

| | Mean value of antibody titers (Log 10 PFU/g) ± Standard deviation | | | |
|---|---|---|---|---|
| | Nasal concha | | Lung | |
| | 3 days after | 6 days after | 3 days after | 6 days after |
| Compounds | infection | infection | infection | infection |
| PBS | 6.6 ± 0.2 | 5.9 ± 0.3 | 7.7 ± 0.1 | 6.7 ± 0.4 |
| Neotame | 6.3 ± 0.1 | 6.2, 5.5, NA | 7.5 ± 0.2 | 7, 6.5, NA |
| Norbixin | 6.7 ± 0.2 | 5.6 ± 0.3 | 7.4 ± 0.3 | 6.4 ± 0.3 |
| γ-Undecalactone | 6.7 ± 0.1 | 6.0 ± 0.3 | 7.4 ± 0.2 | 6.2 ± 0.2 |
| Only vaccine | 6.3 ± 0.2 | 5.4 ± 0.7 | 7.4 ± 0.2 | 5.7 ± 1.0 |
| Vaccine + Neotame | 6.1 ± 0.05 | 3.3 ± 0.4 | 7.5 ± 0.3 | ND, 5.9, 4.4 |
| Vaccine + Norbixin | 6.0 ± 0.3 | 4.2 ± 1.1 | 7.2 ± 0.3 | 4.4 ± 1.0 |
| Vaccine + γ-Undecalactone | 6.2 ± 0.2 | 4.8 ± 0.3 | 7.0 ± 0.3 | 4.4 ± 1.2 |
| Vaccine + alum | 6.3 ± 0.1 | 3.6 ± 0.7 | 7.4 ± 0.3 | ND, 2.8, ND |

NA: Not assayed (since the mice died 5 days after infection)
ND: Not detected

TABLE 9

| | Mean value of antibody titers (Log 10 PFU/g) ± Standard deviation | | | |
|---|---|---|---|---|
| | Nasal concha | | Lung | |
| | 3 days after | 6 days after | 3 days after | 6 days after |
| Compounds | infection | infection | infection | infection |
| PBS | 6.4 ± 0.1 | 5.4, NA, 5.3 | 6.9 ± 0.04 | 6.0, NA, 5.7 |
| EMANON CH-25 | 6.4 ± 0.1 | 5.3 ± 0.2 | 7.1 ± 0.1 | 5.8 ± 0.2 |
| EMANON CH-60K | 6.4 ± 0.1 | 5.5, NA, 6.2 | 7.1 ± 0.2 | 5.8, NA, 5.8 |
| Hydroxypropyl cellulose | 6.6 ± 0.2 | 5.9 ± 0.02 | 7.0 ± 0.1 | 6.0 ± 0.1 |
| Sodium benzoate | 6.4 ± 0.1 | 5.0 ± 0.6 | 6.7 ± 0.2 | 6.2 ± 0.2 |
| Sodium sulfite | 6.1 ± 0.3 | 5.5 ± 0.1 | 7.0 ± 0.1 | 5.8, NA, 6.3 |
| Only vaccine | 6.3 ± 0.1 | 4.3 ± 0.8 | 6.9 ± 0.1 | 4.9 ± 0.9 |
| Vaccine + EMANON CH-25 | 6.2 ± 0.1 | 3.3 ± 1.0 | 7.1 ± 0.1 | 4.0, 4.9, ND |
| Vaccine + EMANON CH-60K | 6.1 ± 0.2 | 1.7, 3.6, ND | 7.1 ± 0.2 | ND, 5.1, ND |
| Vaccine + Hydroxypropyl cellulose | 6.5 ± 0.1 | 4.3, ND, ND | 7.0 ± 0.2 | 5.9, ND, ND |
| Vaccine + Sodium benzoate | 6.2 ± 0.04 | 5.5, 4.3, ND | 7.0 ± 0.2 | 5.1 ± 1.2 |
| Vaccine + Sodium sulfite | 6.2 ± 0.2 | 4.1 ± 0.5 | 7.0 ± 0.1 | 4.4 ± 0.9 |
| Vaccine + alum | 6.5 ± 0.3 | 3.7, ND, NA | 6.9 ± 0.1 | 3.4, ND, NA |

NA: Not applied (since the mice died 5 days after infection)
ND: Not detected

### 2-4. Effects as Nasal (Mucosal) Vaccine Adjuvants

The effects of the adjuvant candidate compounds, which had been confirmed to have adjuvant effects when administered via intramuscular injection, as nasal vaccine adjuvants were examined.

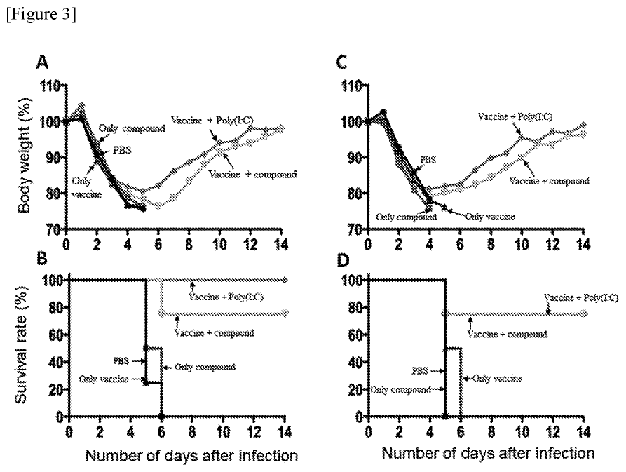

The results of calcium glycerophosphate hydrate and rutin hydrate are shown in FIGS. 3(A and C). The influence of both of these compounds on body weight was the same level as the previously known adjuvant Poly(I:C), and thus, it is considered that these compounds would not affect the body weight of the mice. Also regarding the survival rate (B: calcium glycerophosphate hydrate, D: rutin hydrate), the influence of these compounds was the same level as Poly(I:C) or was slightly lower than Poly(I:C), and thus, it is considered that the compounds would sufficiently have the function of nasal vaccine adjuvants.

The top 8 compounds (including calcium glycerophosphate hydrate and rutin hydrate) that exhibited favorable effects as nasal vaccine adjuvants (in terms of antibody titer and survival rate) are summarized in Table 10.

TABLE 10

| | Antibody | Survival | |
|---|---|---|---|
| Compounds | titers (IgG) | rate (%) | Dose |
| Calcium glycerophosphate hydrate | 261.25 | 75 | 100 μg/dose |
| Rutin hydrate | 280 | 75 | 100 μg/dose |
| Sepiolite | 5440 | 50 | 100 μg/dose |
| Norbixin | 340 | 50 | 100 μg/dose |
| β-D-Glucan | 885 | 50 | 100 μg/dose |
| Riboflavin | 880 | 25 | 100 μg/dose |
| Crocin | 160* | 50 | 100 μg/dose |
| Saponin | 5440 | (100) | 10 μg/dose |
| Poly(I:C) | | | 10 μg/dose |

*One out of four immunized mice had an antibody titer of 160. Other mice had an antibody titer of <10.

### 2-4. Effects of Adjuvants in Ebola Virus Vaccine

From among the food additive-derived compounds and the injection additive-derived compounds identified by the secondary screening, EMANON CH-60K, hydroxypropyl cellulose, polyoxyethylene polyoxypropylene glycol)(160E.O.) (30P.O.), calcium glycerophosphate hydrate, sodium chondroitin sulfate, and riboflavin were selected, and the effects of these compounds as adjuvants for the Ebola virus vaccine were then examined. So far, the effects of EMANON CH-60K and hydroxypropyl cellulose as adjuvants have never been reported, and polyoxyethylene polyoxypropylene glycol)(160E.O.) (30P.O.), calcium glycerophosphate hydrate, and sodium chondroitin sulfate have been reported to have the effects of adjuvants for vaccines other than influenza virus vaccine, and further, flavin has been reported to have the effects of an adjuvant for the influenza virus vaccine. An MF59-like compound was used as a positive control adjuvant.

It was confirmed that the novel adjuvant candidate compounds, EMANON CH-60K and hydroxypropyl cellulose, induce a higher antibody titer against the Ebola GP protein than the MF-59-like compound does, and that other adjuvant candidate compounds, namely, polyoxyethylene polyoxypropylene glycol (160E.O.) (30P.O.), calcium glycerophosphate hydrate, sodium chondroitin sulfate, and riboflavin also exhibit the same effects as those described above (FIG. 1 and FIG. 2).

The top 9 compounds having a high antibody titer induced against the GP protein are summarized in Table 11.

TABLE 11

| | Mean value of antibody titers | | | | |
|---|---|---|---|---|---|
| | Only | Only | Vaccine + | Vaccine + | Antibody titer |
| Compounds | compound (c) | vaccine | compound | AddaVax | ratio (a) |
| EMANON CH-25 | <10 | 20, 80, 20 (b) | 400.00 | 600.00 | 0.67 |
| EMANON CH-40 | <10 | 20, 80, 20 | 300.00 | 600.00 | 0.50 |
| EMANON CH-60K | <10 | 20, 80, 20 | 800.00 | 600.00 | 1.33 |
| Ethanol | <10 | 20, 80, 20 | 140.00 | 600.00 | 0.23 |
| Hydroxypropyl cellulose | <10 | 20, 80, 20 | 480.00 | 600.00 | 0.80 |
| Polyethylene glycol 4,000 | <10 | 20, 80, 20 | 240.00 | 600.00 | 0.40 |
| Polyoxyethylene polyoxypropylene | <10 | 20, 80, 20 | 340.00 | 600.00 | 0.57 |
| glycol (160E.O.) (30P.O.) | | | | | |
| RHEODOL AO-15V | <10 | 20, 80, 20 | 240.00 | 600.00 | 0.40 |
| Zinc oxide | <10 | 20, 80, 20 | 400.00 | 600.00 | 0.67 |

(a) [Mean value of antibody titers (VLP + candidate compound)]/[Mean value of antibody titers (VLP + AddaVax)]
(b) The antibody titer could be measured in some of the 4 mice used in the experiment, but the antibody titer of other mice was <10.
(c) The mice in the group inoculated with only the candidate compound were treated in the same manner as in the case of screening influenza vaccine adjuvant dandidate compounds.

The compounds hit by the method for screening adjuvants disclosed in the present Examples include several compounds whose adjuvant effects have already been reported. Hence, it is suggested that the present screening method be an effective method for screening adjuvant candidate compounds.

Moreover, several compounds identified by the present screening exhibit the effects of Ebola virus vaccine adjuvants. Accordingly, the 34 novel adjuvant candidate compounds according to the present invention are considered to be adjuvant candidate compounds for various virus vaccines.

### INDUSTRIAL APPLICABILITY

The adjuvant candidate compounds according to the present invention have been approved in terms of the safety thereof, and have the effect of sufficiently enhancing immune function. Thus, it is expected that the present adjuvant candidate compounds will be utilized in the field of immunotherapy.

1. An adjuvant comprising one or more selected from the group consisting of hydroxypropyl cellulose, sodium benzoate, and sodium sulfite.

2. A vaccine comprising the adjuvant according to claim 1 and an antigen.

3. The vaccine according to claim 2, wherein the antigen is at least one selected from the group consisting of: viruses, bacteria, parasites, fungi, rickettsiae, chlamydia, prions and cancer cells, and molecules derived from these; cancer antigens; autoimmune disease-related antigens; and allergy-related antigens.
